(19) United States
(12) Patent Application Publication
(10) Pub. No.: US 2018/0104108 A1
(43) Pub. Date: Apr. 19, 2018

(54) COMPRESSION SLEEVE AND MATERIAL USED THEREIN

(71) Applicant: Implus Footcare, LLC, Durham, NC (US)

(72) Inventor: John Angelo Carpinelli, Cary, NC (US)

(73) Assignee: Implus Footcare, LLC, Durham, NC (US)

(21) Appl. No.: 15/293,168

(22) Filed: Oct. 13, 2016

(51) Int. Cl. A61F 13/00; A61F 5/01

(52) U.S. Cl. CPC A61F 13/00017; A61F 5/0109; A61F 2013/0028; A61F 13/00029; A61F 13/00038

(57) ABSTRACT

Compression sleeve fabricated of a composite material that surrounds the joint to be supported and has a main layer of elastically stretchable material and at least one thin film of stretchable material which is laminated to the main layer and provides additional compressive forces that combine with compressive forces provided by the main layer in supporting a joint about which the sleeve is stretched. The material has a third layer which in some embodiments is a nylon jersey material, while in others it is another stretchable material that provides additional compressive forces and support.

### BACKGROUND OF THE INVENTION

### Field of Invention

This invention relates generally to supporting sleeves and wraps for the joints of a person and, more particularly, to a compression sleeve and material for use in a compression sleeve.

### Related Art

Compression sleeves and wraps provide orthopedic support for joints such as the knee, ankle, elbow, and wrist during weightlifting and other activities. Such devices are typically made of an elastic material such as neoprene that surrounds the joint and applies a compressive force to the joint when it is stretched.

However, sleeves made of neoprene tend to be rather bulky and can bunch up or gather on the inner side of a joint, in which case they no longer provide compressive support in that area.

### OBJECTS AND SUMMARY OF THE INVENTION

It is, in general, an object of the invention to provide a new and improved compression sleeve and material for use therein.

Another object of the invention is to provide a compression sleeve and material of the above character which overcome the limitations and disadvantages of compression sleeves and materials heretofore provided.

These and other objects are achieved in accordance with the invention by providing a compression sleeve fabricated of a composite material that surrounds the joint to be supported and has a main layer of elastically stretchable material and at least one thin film of stretchable material which is laminated to the main layer and provides additional compressive forces that combine with compressive forces provided by the main layer in supporting a joint about which the sleeve is stretched. The material has a third layer which in some embodiments is a nylon jersey material, while in others it is another stretchable material that provides additional compressive forces and support.

### BRIEF DESCRIPTION OF THE DRAWINGS

FIG. 1 is a perspective view, partly broken away, of embodiment of a composite material for use in compression supports in accordance with the invention.

FIG. 2 is a front elevational view of one embodiment of a compression sleeve fabricated of the composite material of FIG. 1.

FIG. 3 is a side elevational view of the embodiment of FIG. 2.

FIG. 4 is a rear elevational view of the embodiment of FIG. 2.

FIG. 5 is a front elevational view, partly broken away, of another embodiment of a compression sleeve fabricated of the composite material of FIG. 1.

FIG. 6 is a side elevational view, partly broken away, of the embodiment of FIG. 5.

FIG. 7 is a rear elevational view of the embodiment of FIG. 5.

### DETAILED DESCRIPTION

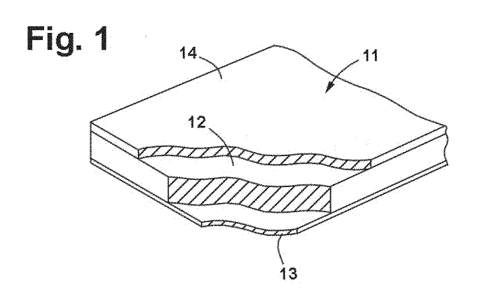

As illustrated in FIG. 1, the material 11 from which the sleeves or wraps of the invention are made is a composite, tri-layer material that includes a central main layer or core 12 of stretchable, elastic material such as neoprene or styrene-butadiene rubber (SBR) having a thickness on the order of 2-3 mm. A thin, stretchable film or foil 13 of polyurethane or similar material is laminated to one side of the main layer or core, and a layer of flexible fabric 14 such as a nylon jersey material is laminated to the other. The combination of the stretchable film and the dominant layer of neoprene or SBR provides greater compression with a much thinner material than the materials heretofore employed in such devices.

Other suitable materials for use in the elastic main layer or core 12 include breathable and non breathable chloroprene rubber (CR), neoprenes, and similar elastomers with the ability to stretch and rebound and exhibit little or no compression set. Another possibility is ethylene vinyl acetate (EVA) memory foam. Other suitable materials for use in the thin stretchable film layer 13 include polyethylene, polyester, elastane, and other similar materials with elastomeric characteristics. Other flexible materials that can be used in outer layer 14 include polyester, elastane, and the like.

If even more compression is desired, layer 14 can also be a stretchable material such as polyurethane, rather than nylon or other fabric.

The composite, tri-layer material is manufactured in sheet form and cut into sections or panels that are sewn or otherwise joined together to make the sleeve, with the contour or configuration of the sleeve being determined by the contours of the panels and the manner in which they are joined together.

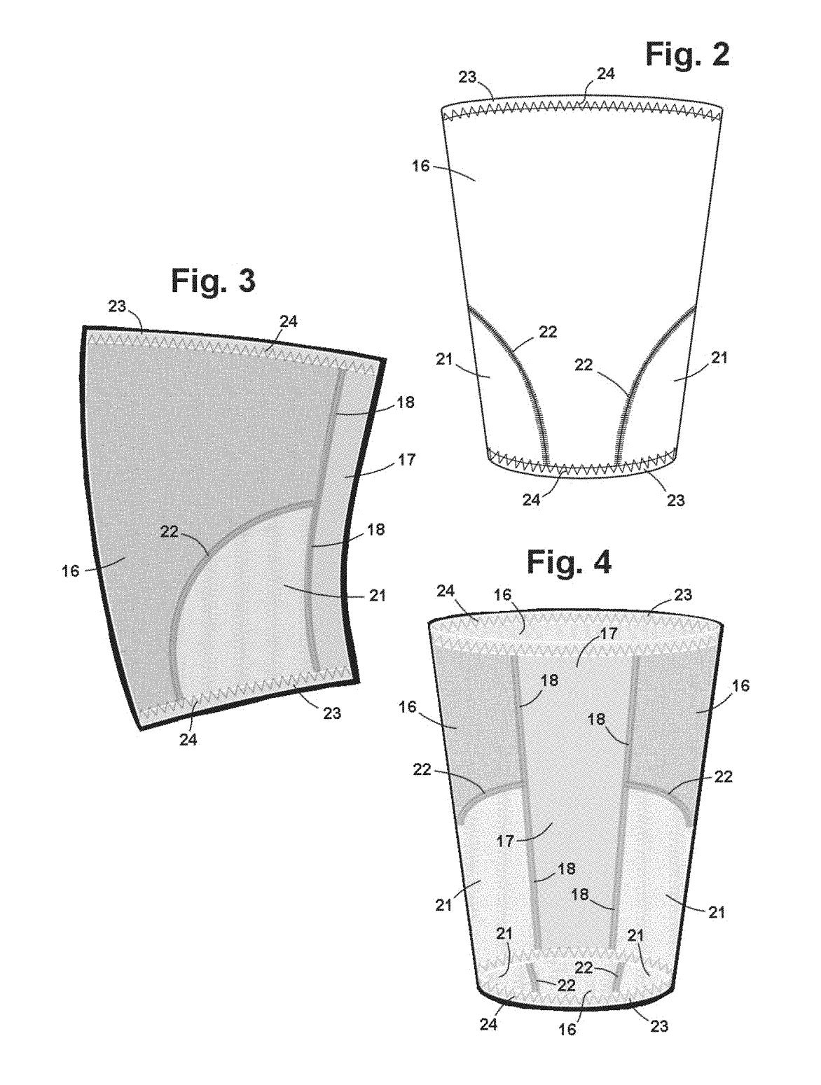

The compression sleeve shown in FIGS. 2-4 is specifically designed to be worn on the knee, but the invention also encompasses sleeves or wraps for other joints such as the ankle, wrist, and elbow. The sleeve has a front panel 16 which goes on the front or anterior side of the knee and a rear panel 17 which goes on the rear or posterior side. The two panels are fabricated of the composite material of FIG. 1 and joined together along their side edges by flat seam stitching 18 to form a tubular body or sleeve.

On the sides of the sleeve, the lower, rear corner portions of front panel 16 are cut away, and replaced with sector-shaped Inserts 21 of another stretchable material, with one edge of the inserts extending along the lower edge of the sleeve and another extending vertically and being attached to the lower portion of rear panel 17 by stitching 18. The curved edge of each insert is attached to front panel 16 by flat seam stitching 22. In one exemplary embodiment, the inserts are fabricated of a synthetic rubber material such as that marketed under the Ariaprene Pro trademark, and have a thickness of 1.0 mm or less. The Ariaprene Pro material is a 3 layer material with a neoprene foam center and outer layers of a material such as nylon. However, the inserts can also be fabricated of polyester, polyester-polyurethane, elastane, or other similar materials with good elasticity.

The upper and lower edges of the sleeve are bound with stretchable nylon binding tape 23 which is secured to the upper and lower margins of panels 16, 17 and to the lower margin of lateral inserts 21 by zig-zag stitching 24.

In one presently preferred embodiment, the composite material of which panels 16, 17 are made has a CR grade neoprene main layer or core 12, with a polyurethane film or foil 13 on one side of the main layer or core and nylon jersey 14 on the other. In this particular embodiment, the neoprene layer is 3.0 mm thick, and the overall thickness of the material is 3.85-4.10 mm.

When the sleeve is manufactured, the composite material is oriented with the nylon jersey on the inside, next to the skin, and the polyurethane on the outside. However, the sleeve is designed to be reversible and, can be worn either the way it is made or inside out with the polyurethane on the inside and the nylon jersey on the outside.

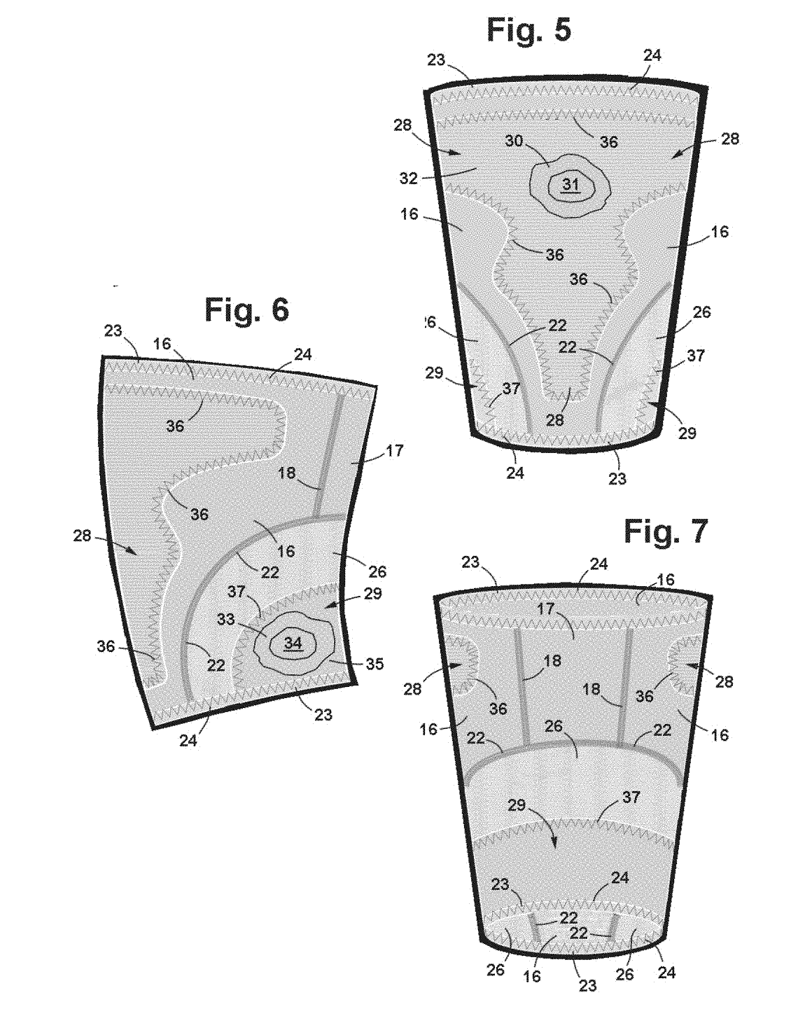

The support sleeve shown in FIGS. 5-7 is generally similar to the embodiment of FIGS. 2-4, and like reference numerals designate corresponding elements in the two embodiments. It differs, however, in that rear panel 17 and flat seam stitching 18 extend only about half way down the back side of the sleeve, and lateral inserts 21 are replaced by a single panel 26 of stretchable material which extends across the back side of the sleeve and is attached to the lower edge of rear panel 17 by a continuation of flat seam stitching 22.

This embodiment also has patches 28, 29 of elastically stretchable material on the front and rear sides of the sleeve for applying additional compressive support to selected areas of the joint on which the sleeve is worn. These patches are fabricated of a composite material similar to material the panels 16, 17 are made of, with patch 28 having a central main layer or core 30, and inner layer 31, and an outer layer 32 and patch 29 having a central main layer or core 33, and inner layer 34, and an outer layer 35.

Patch 28 has a shape generally similar to front panel 16, but is smaller in size. It is attached to the front panel by zig-zag stitching 31 which extends about the periphery of the patch.

Patch 29 has a shape generally similar to stretchable panel 26, but is smaller in size. It is attached to the lower margin of panel 26 by the zig-zag stitching 24 that secures the binding tape and by additional zig-zag stitching 32 which extends along the upper margin of the patch.

As in the embodiment of FIGS. 2-4, the composite material of which front and rear panels 16, 17 are fabricated can, for example, have a neoprene core thickness of 3.0 mm and an overall thickness of 3.85-4.10 mm, whereas the material from which patches 28, 29 are made is somewhat thinner, with a core thickness of 2.0 mm and an overall thickness of 2.85-3.10 mm in this example.

The invention has a number of important features and advantages. It provides a new and improved orthopedic support and a new and improved material for use in such supports. The material is highly elastic and relatively thin, and a compression sleeve or wrap fabricated of it conforms closely to a joint on which its worn and applies a uniform compressive force to the joint.

It is apparent from the foregoing that a new and improved compression support and material been provided. While only certain presently preferred embodiments have been described in detail, as will be apparent to those familiar with the art, certain changes and modifications can be made without departing from the scope of the invention as defined by the following claims.

1. A compression sleeve for supporting a joint of a person, fabricated of a composite material that surrounds the joint and has a main layer of elastically stretchable material and at least one thin film of stretchable material which is laminated to the main layer and provides additional compressive forces that combine with compressive forces provided by the main layer in supporting a joint about which the sleeve is stretched.

2. The compression sleeve of claim 1 wherein the elastically stretchable material in the main layer is neoprene.

3. The compression sleeve of claim 1 wherein the elastically stretchable material in the main layer is styrene-butadiene rubber (SBR).

4. The compression sleeve of claim 1 wherein the film which is laminated to the main layer is polyurethane.

5. The compression sleeve of claim 1 wherein the main layer has a thickness on the order of 2-3 millimeters.

6. The compression sleeve of claim 1 wherein the composite material includes a layer of flexible fabric which is laminated to a side of the main layer opposite the film of stretchable material.

7. The compression sleeve of claim 6 wherein the flexible fabric is nylon.

8. The compression sleeve of claim 1 wherein the composite material includes a second layer of stretchable material which is laminated to the main layer and provides additional compressive forces that add to the compressive forces provided by the main layer and the film of stretchable material.

9. The compressive sleeve of claim 8 wherein the stretchable material in the second layer is selected from the group consisting of spandex and elastene.

10. The compression sleeve of claim 1 wherein the sleeve has front and rear sections of the composite material and at least one additional section of another stretchable material between portions of the front and rear sections.

11. The compression sleeve of claim 10 having lateral inserts of the other stretchable material between portions of the front and rear sections of the composite material on opposite sides of the sleeve.

12. The compression sleeve of claim 10 wherein the rear section of the composite material extends only part way down the rear of the sleeve, and a panel of the other stretchable material extends rearwardly from lower portions of the front section and around the rear of the sleeve below the rear section.

13. The compression sleeve of claim 10 wherein the other stretchable material is synthetic rubber.

14. The compression sleeve of claim 1 further comprising patches of elastically stretchable material on the sleeve for applying additional compressive support to selected areas of the joint about which the sleeve is stretched.

15. The compression sleeve of claim 14 wherein the patches of elastically stretchable material are fabricated of a composite material similar to the first named composite material.

16. The compression sleeve of claim 15 wherein the patches have a main layer of elastically stretchable material that is thinner than the main layer of the first named composite material.

17. The compression sleeve of claim 16 wherein the main layer of the first named composite material has a thickness on the order of 3 mm, and the main layer of the patches has a thickness on the order of 2 mm.

18. A composite material for use in a compression sleeve for supporting a joint of a person, comprising a main layer of elastically stretchable material and at least one thin film of stretchable material which is laminated to the main layer and provides additional compressive forces that combine with compressive forces provided by the main layer in supporting a joint about which the sleeve is stretched.

19. The composite material of claim 18 wherein the elastically stretchable material in the main layer is neoprene.

20. The composite material of claim 18 wherein the elastically stretchable material in the main layer is styrene-butadiene rubber (SBR).

21. The composite material of claim 18 wherein the film which is laminated to the main layer is polyurethane.

22. The composite material of claim 18 wherein the main layer has a thickness on the order of 3 millimeters.

23. The composite material of claim 18 further comprising a layer of flexible fabric which is laminated to a side of the main layer opposite the film of stretchable material.

24. The composite material of claim 21 wherein the flexible fabric is nylon.

25. The composite material of claim 18 further comprising a second layer of stretchable material which is laminated to the main layer and provides additional compressive forces that add to the compressive forces provided by the main layer and the film of stretchable material.

26. The composite material of claim 25 wherein the stretchable material in the second layer is spandex.

27. The composite material of claim 25 wherein the stretchable material in the second layer is elastene.

28. A composite material for use in a compression sleeve for supporting a joint of a person, comprising a main layer of elastically stretchable material selected from the group consisting of neoprene and styrene-butadiene rubber (SBR) having a thickness on the order of 2-3 mm, a thin film of polyurethane laminated to one side of the main layer, and a layer of nylon fabric laminated to the other side of the main layer.

29. A composite material for use in a compression sleeve for supporting a joint of a person, comprising a main layer of elastically stretchable material selected from the group consisting of neoprene and styrene-butadiene rubber (SBR) having a thickness on the order of 2-3 mm, a thin film of polyurethane laminated to one side of the main layer, and a layer of stretchable material such as spandex and elastene laminated to the other side of the main layer.
